(19) United States
(12) Patent Application Publication
(10) Pub. No.: US 2012/0058525 A1
(43) Pub. Date: Mar. 8, 2012

(54) METHOD FOR PRODUCING OIL BY YEAST

(75) Inventors: JYH-YIH LEU, New Taipei (TW); Yu-Sheng Wu, New Taipei (TW)

(73) Assignee: Fu Jen Catholic University, New Taipei City (TW)

(21) Appl. No.: 13/224,326

(22) Filed: Sep. 1, 2011

(30) Foreign Application Priority Data
Sep. 3, 2010 (TW) 099129970

(51) Int. Cl. C12P 7/64; C12N 1/16

(52) U.S. Cl. 435/134; 435/255.1

(57) ABSTRACT

A method for producing oil by using yeast is provided. The method comprises utilizing at least one yeast strain serving as a working microorganism to produce the oil, wherein the yeast strain is genus Pseudozyma.

### CROSS-REFERENCE TO RELATED APPLICATION AND CLAIM OF PRIORITY

The application claims the benefit of Taiwan Patent Application No. 099129970, filed on Sep. 3, 2010, in the Taiwan Intellectual Property Office, the disclosures of which are incorporated herein in their entirety by reference.

### FIELD OF THE INVENTION

The present invention relates to a method for producing oil by using microorganism and the applications thereof, and more particularly to method for producing oil by using yeast and the applications thereof.

### BACKGROUND OF THE INVENTION

Utilizing microorganisms to produce oil is not a recent idea. Besides the microalgae and the bacteria, there are oil-generating microorganisms in fungi, most of which are yeast and mould, and some microorganisms simultaneously have the characteristic of generating long-chain saturated fatty acids, monounsaturated fatty acids or polyunsaturated fatty acids. These oil-generating yeasts can produce fatty acids occupied over 40% of biomass and accumulated up to 70% via the limitation and allocation of the medium, which have rather high potential for producing oil. The following table shows common oil-generating fungi and the oil quantity produced thereby:

| Fungi | Lipid % of dry weight (w/w) |
|---|---|
| Aspergillus terreus | 64 |
| Cryptococcus curvatus | 58 |
| Cryptococcus albidus | 65 |
| Candida sp. | 42 |
| Cunninghamella japonica | >43.8 |
| Lipomyces starkeyi | 63 |
| Penicillium spmulosum | 64 |
| Rhodosporidium toruloides | 56.5 |
| Rhodotorula glutinis | 72 |
| Rhodotorula graminis | 36 |
| Rhizopus arrhizus | 57 |
| Schizochytrium spp. | 30~50 |
| Thraustochytrium spp. | 30~50 |
| Trichosporon pullulans | 65 |
| Yarrowia lipolytica | 36 |

### SUMMARY OF THE INVENTION

In accordance with one aspect of the present invention, a method for producing oil by using yeast is provided. The method comprises utilizing at least one yeast strain serving as a working microorganism to produce the oil, wherein the yeast strain is genus Pseudozyma.

In one embodiment of the present invention, the method further comprises steps of providing the working microorganism a carbon source and a nitrogen source. In one embodiment of the present invention, the nitrogen source is selected from a group consisting of tryptone, gelatin, peptone, yeast extract, soytone and the arbitrary combination thereof. In one embodiment of the present invention, the carbon source is selected from a group consisting of glucose, fructose, maltose, lactose, sucrose, glycerol and the arbitrary combination thereof.

In one embodiment of the present invention, the oil comprises a medium-chain fatty acid (MCFA) having 14 to 20 carbon atoms (referring as C14-C20 series). In one embodiment of the present invention, the oil comprises saturated fatty acids and unsaturated fatty acids suitable for manufacturing bio-diesel oil.

In one embodiment of the present invention, the yeast is selected from a group consisting of Pseudozyma antarctica, P. rugulosa, P. fusiformata, P. hubeiensis, P. flocculosa, P. prolifica, P. pruni and the arbitrary combination thereof.

In one embodiment of the present invention, the cultural temperature of the yeast substantially ranges from 20° C. to 30° C. In one embodiment of the present invention, the cultural pH value of the yeast substantially ranges from 6 to 8.

In accordance with another aspect of the present invention, an oil generation system is provided. The oil generation system comprises a carbon source and at least one yeast strain reacted with the carbon source to produce the oil, wherein the yeast strain is genus Pseudozyma.

Based on the aforementioned embodiments, at least one yeast strain of genus Pseudozyma is utilized for serving as the working microorganism to produce oil. In comparison with the prior art, the oil produced by the yeast strain is less easily oxidized and thus is more suitable for the manufacture of bio-diesel oil.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 illustrates the results of the cultural temperature test for Pseudozyma pruni BCRC 34227;

FIG. 2 illustrates the results of the cultural pH value test for Pseudozyma pruni BCRC 34227;

FIG. 3 illustrates the results of the cultural carbon source test for Pseudozyma pruni BCRC 4227; and

FIG. 4 illustrates the results of the cultural nitrogen source test for Pseudozyma pruni BCRC 4227.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

Cell Line

The working microorganism used for producing oil comprises at least one yeast strain of genus Pseudozyma. In some embodiments of the present invention, cell lines including Pseudozyma antarctica BCRC 33867, P. rugulosa BCRC 33859, P. fusiformata BCRC 22669, P. hubeiensis BCRC 34122, P. flocculosa BCRC 33999, P. prolifica BCRC 34000, P. pruni BCRC 34227 are purchased from the Bioresource Collection and Research Center (BCRC), Taiwan of the Food Industry Research and Development Institute (FIRDI) serving as the working microorganism to produce oil.

Fatty Acid Extraction

Fatty acid extraction is preformed for each collected cultured medium which has been freeze dried into powder. Since fatty acid in organism exists in the form of triglyceride, which is the form of 3 free fatty acids combined with a glycerol via esterification, thus transesterification is needed. The transesterification is performed for Methanol and triglyceride to form single methyl esters to be analyzed. Because the mechanism of transesterification has been well known by persons skilled in the art, thus the detail step and mechanism thereof will not be redundantly described.

In the process of the extraction, Gas Chromatography (GC) is used to determine the quantity of fatty acid in the microorganisms, but the sample is really detected by a detector. Since the detector itself can not provide a stable detectable result due to the characteristic of GC, an internal standard is added to provide a quantifiable basis. The ratio of the analyte to the internal standard, named Relative Response Factor (RRF), is used to determine the quantity of the sample. Because the sample and the internal standard will present proportional increment and decrement in the detection process, more accurate quantification is available. For the choice of an internal standard, substances appearing in the sample and having a similar structure, chemical property and boiling point to the sample will not be chosen as an internal standard. Most fatty acids existing in organisms are even-carbon chains; odd-carbon chains are very rare. Thus, nonadecanioc acid is used as an internal standard. The extraction steps are as follows.

A. Take 0.05 g dried powder of fungus body into 10 ml tube, add 5 ml chloroform:methanol 2:1 (V/V) and mix well.

B. The fungus body is shaken by ultrasonic waves for 2 min (shake 5 sec and stop 5 sec for totally 4 min)

C. After placed under room temperature for 1 hr, 100 μl 10 mg/ml internal standard is added.

D. Add 0.5 ml water, centrifuged at 2500 rpm for 5 min and then remove the suspension.

E. Add 2.5 ml TUP (theoretical upper phase, chloroform ddH2O:methanol=47:48:3) without shaking, centrifuged at 2500 rpm for 5 min and then remove the suspension.

F. Repeat steps D and E, and dry the precipitate by nitrogen gas under room temperature.

G. Add 2.5 ml methanol-benzene 4:1 (V/V), slowly add 250 μl acetyl chloride as catalyst of transesterification, and mix well.

H. Cover the Teflon lid tightly and place in the 80° C. oven for 4 hr.

I. After cooled under room temperature, add 1.5 ml 7% K₂CO₃ slowly to stop the reaction, and centrifuged at 2500 rpm for 10 min.

J. Take the upper benzene layer to the discarded centrifugal tube, and dry the precipitate by nitrogen gas under room temperature.

K. Add 0.5 ml hexane along the tube wall and mix well.

L. After dried by nitrogen gas, add 250 μl hexane, mix well, and inject into the sample bottle having the insert tube. After sealing the opening, use GC to analyze.

Analyze Fatty Acid by Gas Chromatography (GC)

The preferred chromatographic column is lower polarity DB-1 having a length of 60 m and an inner diameter of 0.25 mm; the inner membrane of the chromatographic column is Dimethylpolysiloxane ([—O—Si(CH₃)₂—]) and the thickness thereof is 0.25 μm; the initial temperature of the oven is 60° C. and heat to 280° C.; the heating temperature of the injector is 250° C.; the flow rate of nitrogen gas is 1.2 ml/min, the flow rate of hydrogen gas is 30 ml/min, and the flow rate of air is 300 ml/min; the injection amount of the sample is 1 μl; the fire ion detector (FID) is used for detecting sample; and the temperature is set to 300° C. The result is integrated by GC kit software, and fatty acid contents are estimated by the standard. The estimated equation is:

Single fatty acid %=(integration area of single fatty acid×concentration of the standard×100)/integration area of the internal standard

Total fatty acid %=[(integration area of total fatty acid−integration area of the internal standard)×concentration of the standard×100]/integration area of the internal standard

Test of the Fatty Acid Contents of Genus Pseudozyma.

Yeast strains of Pseudozyma antarctica BCRC 33867, P. rugulosa BCRC 33859, P. fusiformata BCRC 22669, P. hubeiensis BCRC 34122, P. flocculosa BCRC 33999, P. prolifica BCRC 34000, P. pruni BCRC 34227 are cultured in the GYP medium which comprises 5% glucose, 1% yeast extract and 1% peptone.

The medium broths are placed at 20° C., 150 rpm for 7 days of shaking. The cultured yeast strains are then harvested and dried to obtain dried powder, and the fatty acid extraction is performed for the dried powder. Subsequently, the GC-MS analysis is performed to determine the detailed classification of the extracted fatty acid and the content of each type fatty acid. The results of fatty acid content are shown in the following table

| | Yeast Strain | | | | | | |
|---|---|---|---|---|---|---|---|
| | BCRC | BCRC | BCRC | BCRC | BCRC | BCRC | BCRC |
| | 22669 | 33859 | 33867 | 33999 | 34000 | 34122 | 34227 |
| Type of fatty acid | Content (%) | | | | | | |
| C16:0 | 8.34 | 23.22 | 22.41 | 16.52 | 2.60 | 3.65 | 20.72 |
| C16:1 | 9.16 | 6.06 | 3.72 | 3.09 | 1.57 | 16.40 | 6.94 |
| C18:0 | 7.52 | 6.17 | 8.64 | 3.76 | 1.16 | 28.66 | 7.28 |
| C18:1 | 16.14 | 24.09 | 20.56 | 18.29 | 4.03 | 4.47 | 33.51 |
| C18:2 | 13.31 | 24.79 | 19.33 | 34.47 | 1.86 | 5.55 | 2.47 |
| C18:3 | 8.89 | 3.79 | 4.80 | 7.30 | 43.18 | 13.87 | |
| C20:0 | 4.16 | 2.64 | 6.14 | 5.54 | 11.55 | 7.39 | 2.64 |
| C22:0 | 1.74 | 1.29 | 2.48 | 2.26 | 4.82 | 3.90 | 2.60 |
| Content of the | 26.4 | 36.2 | 28.6 | 15.2 | 22.2 | 29.3 | 45.7 |
| total fatty acid (%) | | | | | | | |

The analysis results indicates that the dried genus Pseudozyma cultured for 7 days contents total fatty acid ranges about 15˜45% by weight. P. pruni BCRC 34227 particularly contents total fatty acid substantially greater than 48%. Thus, the fact that yeast strain of genus Pseudozyma is capable for serving as the working microorganism to produce oil with a high yield rate can be approved.

Besides, as shown in the above, most of fatty acids produced by these yeast strains are MCFA having 14 to 20 carbon atoms, including the saturated fatty acid and unsaturated fatty acid of C16, C18, C20 and C22 series, which are less easily oxidized, in comparison with the polyunsaturated fatty acids produced by the prior art, the fatty acid produced by these yeast strains of genus Pseudozyma are more suitable for the manufacture of bio-diesel oil.

Additionally, the yield of total fatty acid may be improved when these yeast strains of genus Pseudozyma are stimulated by the optimal treatment, so it has high potential for serving as a source of bio-diesel oil. Range test of cultural temperature, optimal pH value test, test of utilizing the nitrogen source and the carbon source are performed for P. pruni BCRC 34227 to find out the optimal parameters of oil generation. However, it should be appreciated that, the testing result is merely illustrative but not intend to limit the present invention, various modifications and similar arrangements included within the spirit may be performed by the persons skilled in the art to find out the optimal parameters of other strains of genus Pseudozyma.

Range Test of Cultural Temperature

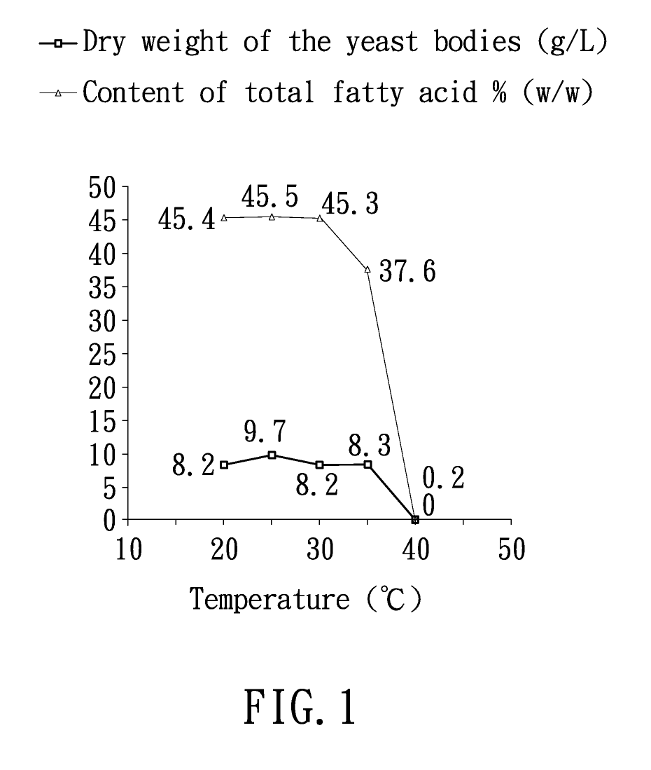

Pseudozyma pruni BCRC 34227 is cultured in 50 ml GYP broth under 5 different temperatures, such as 20° C. [symbol] 25° C. [symbol] 30° C. [symbol] 35° C. [symbol] 40° C., and the yeast bodies are collected and analyzed after culturing for 7 days. The analysis results of dried weight and fatty acid are shown in FIG. 1.

In accordance with FIG. 1, biomass and yield of fatty acid can be improved in the temperature range of 20° C. to 30° C., and there are the best biomass and yield of fatty acid cultured in 25° C.

Optimal pH Value Test

The range of pH values is set as 3-11 to discuss the effect of pH values on the yeast body and the optimal cultural condition. To take out the effect of metabolites generated by the organisms on pH values, based on the GYPG broth, the following buffers are added according to each condition.

| pH value | Buffer and concentration thereof |
|---|---|
| 3-4 | Sodium Acetate, 30 mM |
| 5-6 | MES, 30 mM |
| 7-8 | Tris, 30 mM |
| 9-11 | CAPSO, 30 mM |

The pH value is adjusted by using acetic acid for pH 3-4 and using NaOH or HCl for pH 5-11.

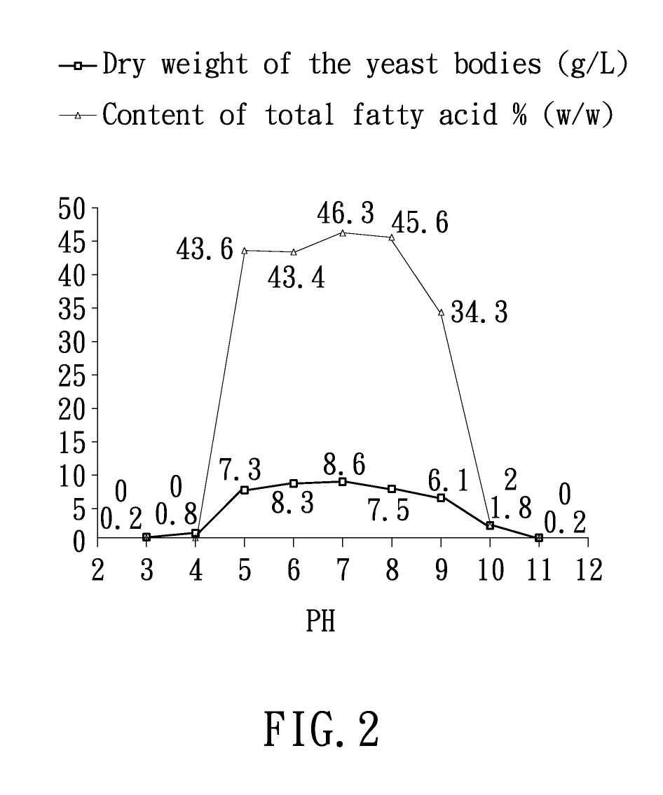

Pseudozyma pruni BCRC 34227 is cultured under each pH value, the optimal temperature and 150 rpm for 7 days. After 7 days, 50 ml broth is collected and dried to determine the dried weight of the yeast bodies and the yield of fatty acid whose results are shown in FIG. 2. In accordance with FIG. 2, biomass and yield of fatty acid can be significantly improved in the pH range of 6 to 8, and the optimal pH value for growth is pH 7.

Test of Utilizing the Carbon Source

The heterotroph needs the carbon source to grow. The utilization of type of carbon source affects the growth of microorganism and the accumulation of oil. According to the metabolic pathway of yeast, various carbon sources including monosaccharide (hexose and pentose), disaccharide, polysaccharide are used, and glycerol is used as the carbon source for oil so as to find the best carbon source for the growth of microorganism and the accumulation of oil. The cultural prescription is as follows.

5% carbon source+0.1% yeast extract+0.1% peptone

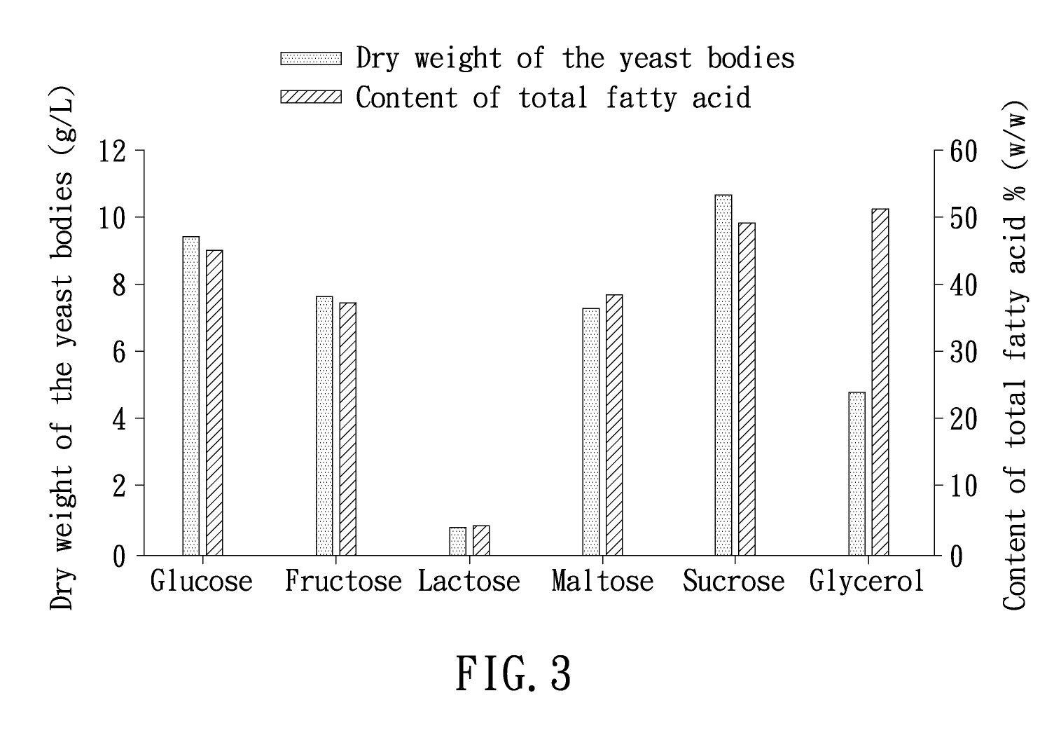

Type of the carbon source: glucose, fructose, lactose, maltose, sucrose and glycerol. Pseudozyma pruni BCRC 34227 is cultured under each carbon source, the optimal temperature and 150 rpm for 7 days. After 7 days, 50 ml broth is collected and dried to determine the dried weight of the yeast bodies and the yield of fatty acid whose results are shown in FIG. 3.

In accordance with FIG. 3, the dried weight and the total fatty acid content are shown in FIG. 3. The carbon source for the best biomass of Pseudozyma pruni BCRC 34227 is sucrose, and the second best is glucose; the carbon source for the best total fatty acid content of Pseudozyma pruni BCRC 34227 is glycerol, and the second best is sucrose.

Test of Utilizing the Nitrogen Source

The nitrogen source is necessary nutrition for the growth of microorganisms. There are researches pointing out that different nitrogen sources affect oil accumulation of microorganisms. Therefore in this case, the organic nitrogen source and the inorganic nitrogen source are designed for observing the condition of the growth of microorganisms and the accumulation of oil so as to find the best nitrogen source. The cultural prescription is as follows.

5% glucose+0.1% yeast extract+0.1% nitrogen source

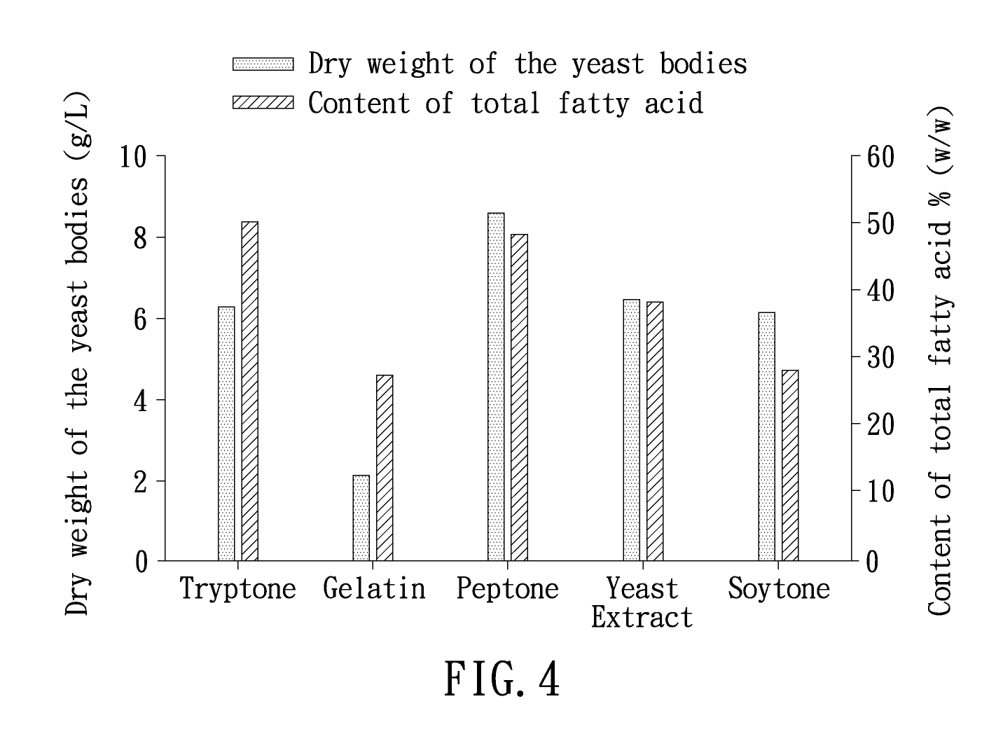

Type of the nitrogen source: tryptone, gelatin, peptone, yeast extract and soytone. Pseudozyma pruni BCRC 34227 is cultured under each nitrogen source, the optimal temperature and 150 rpm for 7 days. After 7 days, 50 ml broth is collected and dried to determine the dried weight of the yeast bodies and the yield of fatty acid whose results are shown in FIG. 4.

In accordance with FIG. 4, the nitrogen source for the best biomass of Pseudozyma pruni BCRC 34227 is peptone, and the nitrogen source for the best total fatty acid content is tryptone.

Based on the aforementioned embodiments, at least one yeast strain of genus Pseudozyma is utilized for serving as the working microorganism to produce oil, wherein the preferred cultural temperature ranges from 20° C. to 30° C.; the preferred cultural pH value substantially ranges from 6 to 8; the nitrogen source for the best biomass and the best total fatty acid content may be peptone or tryptone; and the carbon source for the best biomass and the best total fatty acid content may be glycerol or saccharide.

In comparison with the prior art, the oil produced by the yeast strain is less easily oxidized and thus is more suitable for the manufacture of bio-diesel oil. Accordingly, the present invention effectively solves the problems and drawbacks in the prior art.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for producing oil by using yeast, comprising:
utilizing at least one yeast strain serving as a working microorganism to produce the oil, wherein the yeast strain is genus Pseudozyma.

2. The method according to claim 1, further comprising steps of providing the working microorganism a carbon source and a nitrogen source.

3. The method according to claim 1, wherein the nitrogen source is selected from a group consisting of tryptone, gelatin, peptone, yeast extract, soytone and the arbitrary combination thereof.

4. The method according to claim 1, wherein the carbon source is selected from a group consisting of glucose, fructose, maltose, lactose, sucrose, glycerol and the arbitrary combination thereof.

5. The method according to claim 1, wherein the oil comprises a medium-chain fatty acid (MCFA) having 14 to 20 carbon atoms.

6. The method according to claim 1, wherein the oil comprises saturated fatty acids and unsaturated fatty acids suitable for manufacturing bio-diesel oil.

7. The method according to claim 1, wherein the yeast is selected from a group consisting of Pseudozyma antarctica, P. rugulosa, P. fusiformata, P. hubeiensis, P. flocculosa, P. prolifica, P. pruni and the arbitrary combination thereof.

8. The method according to claim 1, wherein the cultural temperature of the yeast substantially ranges from 20° C. to 30° C.

9. The method according to claim 1, wherein the cultural pH value of the yeast substantially ranges from 6 to 8.

10. An oil generation system, comprising:
a carbon source; and
at least one yeast strain reacted with the carbon source to produce oil, wherein the yeast strain is genus Pseudozyma.
